(19) United States
(12) Patent Application Publication
(10) Pub. No.: US 2026/0174489 A1
(43) Pub. Date: Jun. 25, 2026

(54) CRYOABLATION NEEDLE TO CONSOLE CONNECTOR

(71) Applicant: Boston Scientific Scimed, Inc., Maple Grove, MN (US)

(72) Inventors: Abigail Blanchette Teschendorf, Blaine, MN (US); Andrew Kevin Zachman, St. Michael, MN (US); Mark Edward DiLoreto, Chaska, MN (US)

(21) Appl. No.: 19/334,151

(22) Filed: Sep. 19, 2025

Related U.S. Application Data
(60) Provisional application No. 63/707,344, filed on Oct. 15, 2024.

(51) Int. Cl. A61B 18/02; A61B 17/00; A61B 18/00

(52) U.S. Cl. CPC A61B 18/02; A61B 2017/00199; A61B 2017/00477; A61B 2017/00526; A61B 2018/00178; A61B 2018/00577; A61B 2018/0262

(57) ABSTRACT

Embodiments herein relate to cryoablation systems. In an embodiment, a cryosurgery system is included having a cryoablation probe assembly having a supply tube, and a return tube surrounding the supply tube. The cryosurgery system can include a console having a cryoablation probe port and a console connector configured to sealingly connect the cryoablation probe assembly to the cryoablation probe port. The cryosurgery system can include a console connector having a main body configured to sealingly connect to a proximal end of the return tube, a supply line adaptor configured to sealingly connect to a proximal end of the supply tube, and a clip. The clip can be configured to couple the supply line adaptor to the main body by fastening into a clip receiving structure of an inner surface of the main body.

This application claims the benefit of U.S. Provisional Application No. 63/707,344, filed Oct. 15, 2024, the content of which is herein incorporated by reference in its entirety.

### FIELD

Embodiments herein relate to cryoablation systems and more particularly to console connectors for cryoablation systems.

### BACKGROUND

During cryosurgery, a surgeon may deploy one or more cryoprobes to ablate a target area of a patient anatomy by freezing and thawing the tissue. In one example, a cryoablation probe uses the Joule-Thompson effect to produce cooling or heating of the probe tip. In such cases, the expansion of a cryofluid in the cryoablation probe from a higher pressure to a lower pressure without heat exchange to the environment leads to cooling of the device tip. Heat transfer between the expanded cryofluid and the outer walls of the cryoprobe leads to formation of an ice ball in the tissue around the tip and consequent cryoablation of the tissue.

### SUMMARY

In a first aspect, a cryosurgery system, can be included having a cryoablation probe assembly. The cryoablation probe assembly can include a supply tube, and a return tube surrounding the supply tube. A console can be included having a cryoablation probe port and a console connector configured to sealingly connect the cryoablation probe assembly to the cryoablation probe port of the console. A console connector can be included having a main body configured to sealingly connect to a proximal end of the return tube, a supply line adaptor configured to sealingly connect to a proximal end of the supply tube, and a clip. In various embodiments, the clip can be configured to couple the supply line adaptor to the main body by fastening into a clip receiving structure of an inner surface of the main body.

In a second aspect, in addition to one or more of the preceding or following aspects, or in the alternative to some aspects, the main body can be overmolded onto the proximal end of the return tube.

In a third aspect, in addition to one or more of the preceding or following aspects, or in the alternative to some aspects, the supply line adaptor can be overmolded onto the proximal end of the supply tube.

In a fourth aspect, in addition to one or more of the preceding or following aspects, or in the alternative to some aspects, the console connector can further include a circuit board assembly, wherein the main body can be configured to be overmolded onto one or more wires connected to the cryoablation probe assembly, wherein a proximal end of each of the one or more wires protrudes from the main body and connects to the circuit board assembly.

In a fifth aspect, in addition to one or more of the preceding or following aspects, or in the alternative to some aspects, the console connector further includes an electrical cap configured to cover the circuit board assembly.

In a sixth aspect, in addition to one or more of the preceding or following aspects, or in the alternative to some aspects, the circuit board assembly includes an adaptor configured to electrically connect the one or more wires of the console connector to the cryoablation probe port of the console.

In a seventh aspect, in addition to one or more of the preceding or following aspects, or in the alternative to some aspects, the cryosurgery system can be a closed-loop cryosurgery system.

In an eighth aspect, in addition to one or more of the preceding or following aspects, or in the alternative to some aspects, the console connector further includes a main body gasket, wherein the main body gasket can be configured to form a seal with an inner diameter of the cryoablation probe port.

In a ninth aspect, in addition to one or more of the preceding or following aspects, or in the alternative to some aspects, the console connector further includes a supply line adaptor gasket configured to be positioned at a neck of a supply line adaptor proximal end.

In a tenth aspect, in addition to one or more of the preceding or following aspects, or in the alternative to some aspects, the clip can be configured to fit around a portion of the supply line adaptor in an interference fit.

In an eleventh aspect, in addition to one or more of the preceding or following aspects, or in the alternative to some aspects, the clip receiving structure of the main body includes a cavity defined in the inner surface of the main body.

In a twelfth aspect, in addition to one or more of the preceding or following aspects, or in the alternative to some aspects, the main body and the supply line adaptor include a polymeric material.

In a thirteenth aspect, a console connector configured to sealingly connect a cryoablation probe assembly to a cryoablation probe port of a console is included. The console connector can have a main body configured to sealingly connect to a proximal end of a return tube of the cryoablation probe assembly. The console connector can have a supply line adaptor assembly having a supply line adaptor configured to sealingly connect to a proximal end of a supply tube of the cryoablation probe assembly. The console connector can have a clip, wherein the clip can be configured to couple the supply line adaptor to the main body by fastening into a clip receiving structure of an inner surface of the main body. In various embodiments, the supply tube can be positioned within the return tube when the supply line adaptor can be fit within the main body.

In a fourteenth aspect, in addition to one or more of the preceding or following aspects, or in the alternative to some aspects, the main body can be overmolded onto the proximal end of the return tube and wherein the supply line adaptor can be overmolded onto the proximal end of the supply tube.

In a fifteenth aspect, in addition to one or more of the preceding or following aspects, or in the alternative to some aspects, the main body can be configured to be overmolded onto one or more wires connected to the cryoablation probe, and wherein a proximal end of each of the one or more wires protrudes from the main body and connects to a circuit board assembly.

In a sixteenth aspect, in addition to one or more of the preceding or following aspects, or in the alternative to some aspects, the console connector further includes a main body gasket, wherein the main body gasket can be configured to form a seal with the cryoablation probe port.

In a seventeenth aspect, in addition to one or more of the preceding or following aspects, or in the alternative to some aspects, the supply line adaptor assembly can further include a supply line adaptor gasket configured to engage with a neck feature of a supply line adaptor proximal end.

In an eighteenth aspect, in addition to one or more of the preceding or following aspects, or in the alternative to some aspects, the clip can be configured to fit around a portion of the supply line adaptor in an interference fit.

In a nineteenth aspect, in addition to one or more of the preceding or following aspects, or in the alternative to some aspects, the main body and the supply line adaptor include a polymeric material.

In a twentieth aspect, a method of forming a console connector configured to sealingly connect a cryoablation probe assembly to a cryoablation probe port of a console can be included. The method can include providing a return tube. The method can include forming a main body assembly by overmolding a main body onto a proximal end of the return tube, wherein the main body can be sealingly connected to the proximal end of a return tube. The method can include providing a supply tube configured to fit within the return tube. The method can include forming a supply line adaptor assembly by overmolding a supply line adaptor onto a proximal end of the supply tube. In various embodiments, the supply line adaptor can be sealingly connected to the proximal end of the supply tube. The method can include placing a clip around a portion of the supply line adaptor. The method can include inserting the supply tube into the return tube. The method can include engaging the supply line adaptor assembly with an inner diameter of the main body such that the clip fastens into a clip receiving structure of an inner surface of the main body.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cryosurgery system in accordance with various embodiments herein.

FIG. 2 is a perspective, schematic view of a console connector in accordance with various embodiments herein.

FIG. 3 is an exploded view of the console connector of FIG. 2 in accordance with various embodiments herein.

FIG. 4 is a cross-sectional view of the console connector of FIG. 2 in accordance with various embodiments herein.

FIG. 5 is a perspective, schematic view of a main body of a probe-console connector in accordance with various embodiments herein.

FIG. 6 is a perspective, schematic view of a supply line adaptor of a probe-console connector in accordance with various embodiments herein.

FIG. 7 is a perspective, schematic view of a clip of a probe-console connector in accordance with various embodiments herein.

FIG. 8 is a schematic view of a main body assembly in accordance with various embodiments herein.

FIG. 9 is a perspective, schematic view of a supply line adaptor assembly in accordance with various embodiments herein.

FIG. 10 is a perspective view of a cross-section of a proximal portion of the console connector in accordance with various embodiments herein.

FIG. 11 is a flowchart of a method of forming a console connector configured to sealingly connect a cryoablation probe assembly to a cryoablation probe port of a console in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Cryoablation systems utilize the expansion of a working fluid to generate a cooling effect. The temperature at the tip of the cryoablation probe can reach cryogenic temperatures (˜140 Kelvin) to form an ice ball and ablate a region in a patient's anatomy. In some embodiments, it is desirable to utilize a cryoablation system having a closed-loop working fluid circuit in which the expanded working fluid is recirculated.

To maximize the efficiency of the cryoablation system and preserve the working fluid, the components are configured to minimize the amount of working fluid that escapes from the cryoablation system. The connection of the cryoablation probe to a console of the system can be susceptible to working fluid leaks. Accordingly, a console connector can be provided to form a leak-tight seal between the console and the cryoablation probe.

In various embodiments, a cryosurgery system can include a cryoablation probe assembly having a supply tube, and a return tube surrounding the supply tube. The cryosurgery system can include a console having a cryoablation probe port. The cryosurgery system can include a console connector configured to sealingly connect the cryoablation probe assembly to the cryoablation probe port of the console. The console connector can include a main body configured to sealingly connect to a proximal end of the return tube. The console connector can include a supply line adaptor configured to sealingly connect to a proximal end of the supply tube. The console connector can include a clip configured to couple the supply line adaptor to the main body by fastening into a clip receiving structure of an inner surface of the main body.

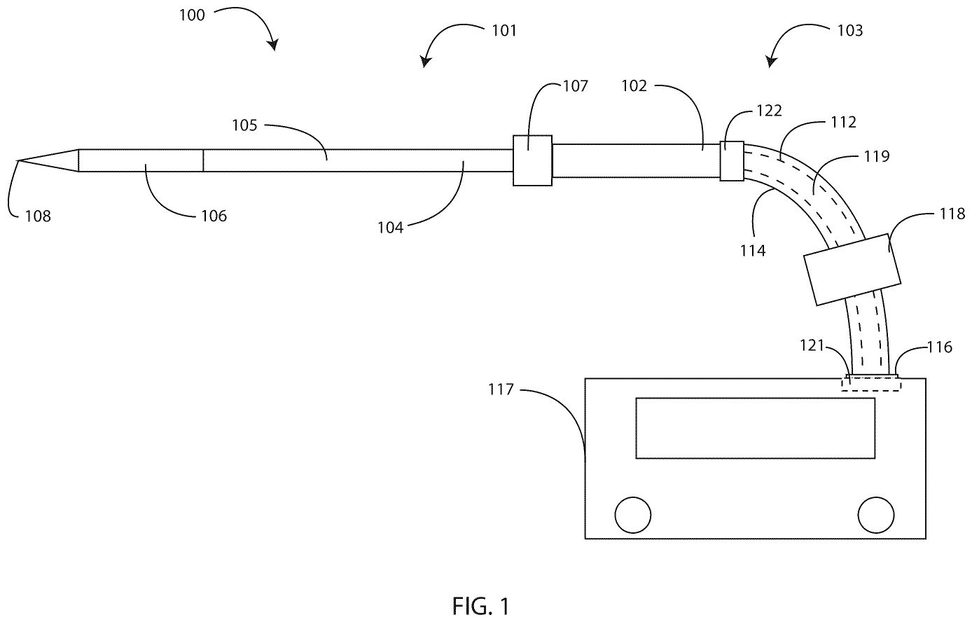

### System Overview (FIG. 1)

Referring now to FIG. 1, a schematic view of a cryosurgery system 100 is shown in accordance with various embodiments herein. In various embodiments, the system can include a cryoablation probe 101 having a handle 102 and a shaft 104. In various embodiments, the shaft 104 is connected to the handle 102 with shaft-handle connector 107. In various embodiments, the shaft 104 and the shaft-handle connector 107 of a cryosurgery system 100 can form a catheter assembly.

In some embodiments, the cryosurgery system 100 includes a console 117. The console may be used to control the system and may be in electrical and fluid communication with the handle and cryoablation assembly. In various embodiments, the console 117 controls the flow of a working fluid. In the example of FIG. 1, the cryosurgery system 100 is a closed-loop system and the working fluid is contained within a closed-loop working fluid circuit within the cryoablation system.

In various embodiments the cryosurgery system 100 can include a cryoablation probe assembly 103. The cryoablation probe assembly 103 can include the cryoablation probe 101 and a line set 119. In various embodiments, line set 119 is configured to connect the console to the handle 102 of the cryoablation probe 101. In various embodiments, the line set 119 is configured to be flexible, enhancing the maneuverability of the cryoablation probe 101. In various embodiments, the line set 119 is formed from a polymeric material or materials including, but not limited to Acrylonitrile Butadiene Styrene (ABS), Nylon, polytetrafluoroethylene (PTFE), one or more polyether block amides (PEBA), or the like. In some embodiments, PEBA can include Pebax®, hereinafter “Pebax” and/or Vestamid®, hereinafter “Vestamid.” Pebax material is available from Arkema Inc., which has a business location at 900 First Avenue, King of Prussia, PA 19406, USA. Vestamid material is available from Evonik Inc., which has a business location at Essen, Rellinghauser Str. 1-11, Germany. In some embodiments, the line set 119 can be reinforced with a braided material, such as a stainless-steel braid, or the like.

In various embodiments, the cryoablation probe assembly 103 is configured to connect to a cryoablation probe port 121 of the console 117. In various embodiments, the cryosurgery system 100 can include a console connector 116 configured to sealingly connect the cryoablation probe assembly 103 to the cryoablation probe port 121 of the console 117. In various embodiments, the cryosurgery system 100 can further include a line set-probe connector 122 configured to form a leak tight seal between the line set 119 and the handle 102 of the cryoablation probe 101.

In the example of FIG. 1, the working fluid is configured to circulate from the console 117 to the cryoablation probe 101 via a line set supply tube 112 and travel back from the cryoablation probe 101 to the console via a line set return tube 114. In the example of FIG. 1, the line set return tube 114 shares a common longitudinal axis with the line set supply tube 112. However, other configurations are possible such as the line set supply tube 112 being arranged adjacent to the line set return tube 114.

In various embodiments, the working fluid circuit runs through both the handle 102 and the shaft 104 of the cryosurgery system 100 and carries the fluid which generates the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. The working fluid is circulated through the probe to generate an ice ball in the patient's body surrounding an expansion chamber 106. The expansion chamber 106 defines the portion of the shaft 104 that is not insulated to increase thermal conduction and where the ice ball is generated. In various embodiments, the shaft 104 carries high pressure working fluid from the handle 102 to the expansion chamber 106, where it undergoes a Joule-Thompson effect and correspondingly experiences a temperature change. The working fluid exits down the shaft 104, through the handle 102, and continues to circulate through the closed-loop working fluid circuit. In various embodiments, the shaft 104 may include an insulated zone 105. The insulated zone 105 defines the portion of shaft 104 that is insulated to reduce thermal losses and define the shape of the ice ball.

The working fluid can be a cooling fluid or mixture of fluids. In some embodiments, the pressure of the high-pressure stream of the working fluid can be greater than or equal to 2.0 MPa, 2.3 MPa, 2.7 MPa, or 3.0 MPa. In some embodiments, the pressure of the high-pressure stream of the working fluid can be less than or equal to 12.0 MPa, 9.0 MPa, 6.0 MPa, or 3.0 MPa. In some embodiments, the pressure of the high-pressure stream of the working fluid can fall within a range of 2.0 MPa to 12.0 MPa, or 2.3 MPa to 9.0 MPa, or 2.7 MPa to 6.0 MPa, or can be about 3.0 MPa. Pressure measurements are provided as absolute pressure measurements herein.

Accordingly, the temperature of the working fluid at the expansion chamber 106 can be about 140 Kelvin. In some embodiments, the temperature of the working fluid at the expansion chamber 106 can be greater than or equal to 100 Kelvin, 113 Kelvin, 127 Kelvin, or 140 Kelvin. In some embodiments, the temperature of the working fluid at the expansion chamber 106 can be less than or equal to 200 Kelvin, 180 Kelvin, 160 Kelvin, or 140 Kelvin. In some embodiments, the temperature of the working fluid at the expansion chamber 106 can fall within a range of 100 Kelvin to 200 Kelvin, or 113 Kelvin to 180 Kelvin, or 127 Kelvin to 160 Kelvin, or can be about 140 Kelvin.

In various embodiments, the cryosurgery system 100 can further include a heat exchanger 118. The heat exchanger 118 can be positioned in any suitable position within the cryosurgery system 100. In the embodiment of FIG. 1, the heat exchanger 118 is disposed in a hub surrounding a line set 119 connecting the cryoablation probe 101 to the console 117. In various embodiments, the heat exchanger 118 is configured to cool the working fluid prior to the working fluid reaching the expansion chamber 106 of the cryoablation probe 101. The heat exchanger 118 can be any suitable heat exchanger such as a recuperative heat exchanger, an electric cooler, or the like.

In some embodiments, the outer surface of the shaft 104 may be thermally insulated from the inner surface of the shaft. In various embodiments, shaft insulation can be provided by a sealed vacuum chamber or containing a non-circulating fluid or gas within a sealed chamber. In alternative embodiments, a vacuum circuit runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft.

The distal end of the shaft 104 may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

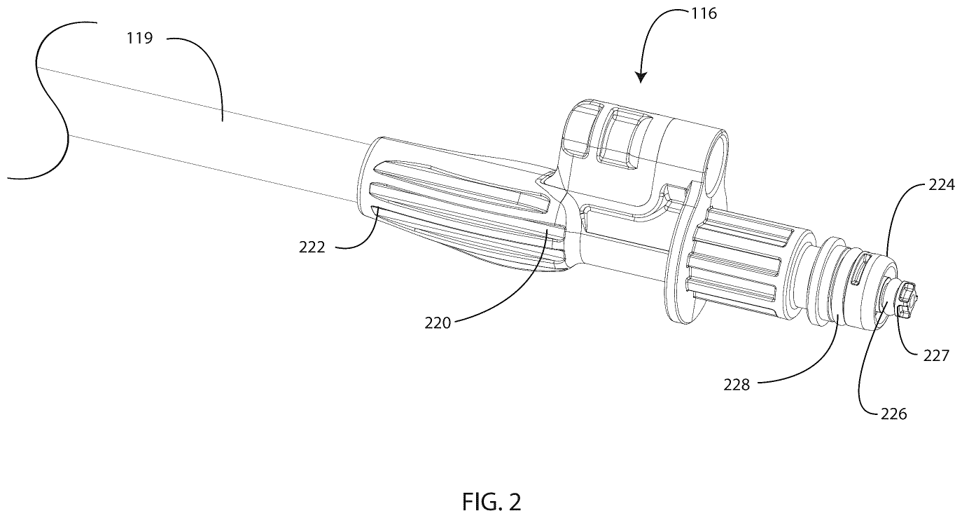

### Console Connector (FIGS. 2-4)

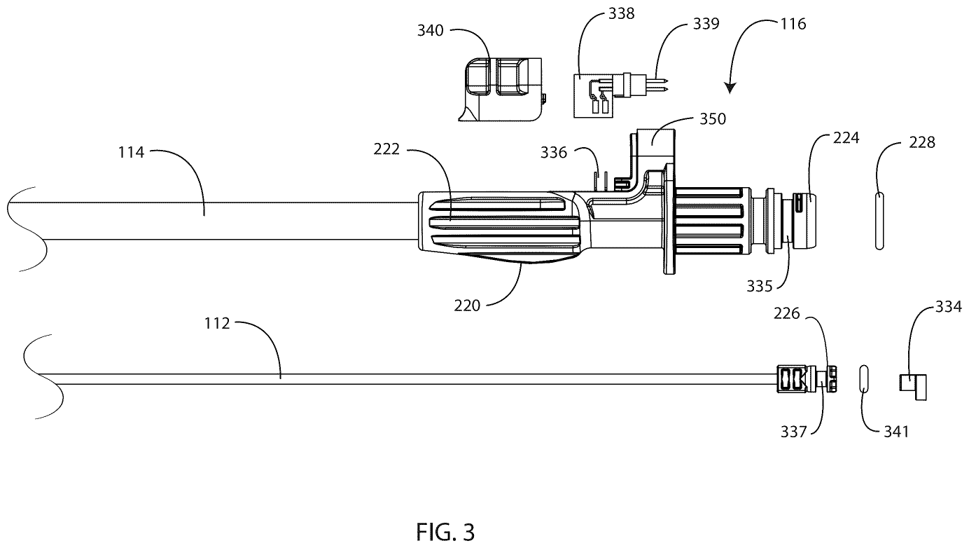

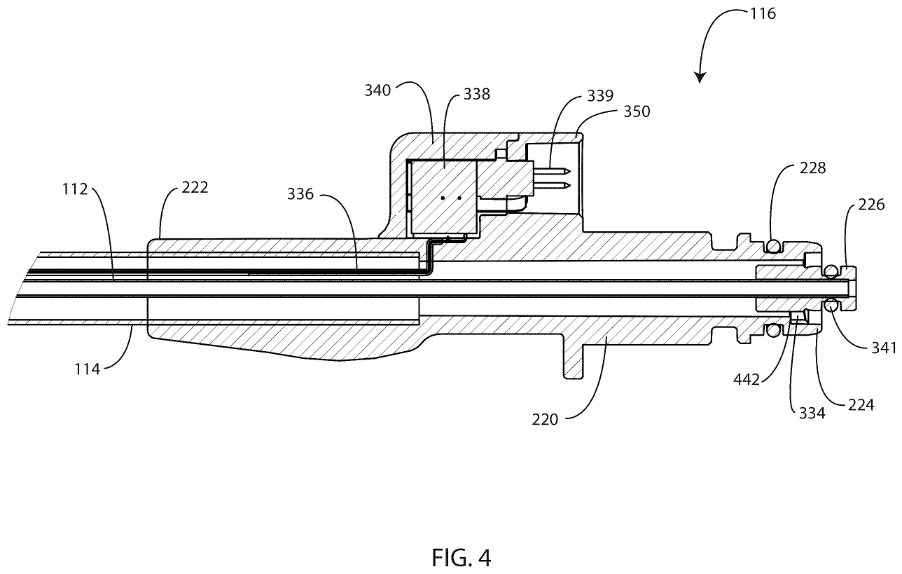

Referring now to FIGS. 2-4, various views of a console connector connected to a line set of a cryoablation probe assembly are shown in accordance with various embodiments herein. FIG. 2 depicts a perspective, schematic view of a console connector in accordance with various embodiments herein. FIG. 3 depicts an exploded view of the console connector of FIG. 2 in accordance with various embodiments herein. FIG. 4 depicts a cross-sectional view of the console connector of FIG. 2 in accordance with various embodiments herein.

As best seen in FIGS. 3-4, the line set 119 includes a supply tube 112 surrounded by a return tube 114. In various embodiments, working fluid is configured to circulate from the console 117 to the cryoablation probe 101 via a line set supply tube 112 and travel back from the cryoablation probe 101 to the console via a line set return tube 114. In various embodiments, the console connector 116 is configured to separate the supply flow of the working fluid from the return flow of the working fluid.

In various embodiments, the console connector 116 is configured to sealingly connect a cryoablation probe assembly 103 to the cryoablation probe port 121 of the console 117. By sealingly connect, it is meant that the console connector 116 is configured to connect the cryoablation probe assembly 103 to the cryoablation probe port 121 of the console 117 such that leaks of the circulating working fluid are minimized and substantially none of the working fluid escapes from the cryosurgery system 100.

In various embodiments, the console connector 116 is configured to removably connect a cryoablation probe assembly 103 to the cryoablation probe port 121 of the console 117. In some embodiments, the cryoablation probe assembly 103 includes the components of the cryosurgery system 100 that are to be replaced each time a cryoablation procedure is performed. In such embodiments, after a cryoablation procedure is performed, a first cryoablation probe assembly having a first console connector can be disengaged from the console and a second cryoablation probe assembly having a second console connector can be plugged into the console, in order to perform a second cryoablation procedure for a different patient.

In various embodiments, the console connector 116 can include a main body 220. In various embodiments, the main body 220 can be formed from a polymeric material or materials, including, but not limited to polyimide, fluorinated ethylene propylene (FEP), Teflon, polytetrafluoroethylene (PTFE), or the like. In some embodiments, the main body 220 can be formed from a resin, such as a Pebax based resin, an ABS based resin, a nylon-based resin, a Vestamid-based resin, or the like. In some embodiments, the resin can contain glass-filled fibers that are configured to reduce shrinking and expansion of the main body 220 caused by temperature fluctuations during a cryoablation procedure. The main body 220 can include a main body distal end 222 configured to sealingly connect to a proximal end of the return tube 114 line set 119.

In some embodiments, the main body 220 is configured to be overmolded onto the proximal end of the return tube 114. Overmolding, as defined herein refers to molding a first component (e.g., the main body 220) over or around a second component (e.g., the return tube 114) using any suitable manufacturing process such as injection molding, or the like. In various embodiments, overmolding the main body 220 to the return tube 114 provides a leak-tight seal and prevents the working fluid from leaking at the interface between the line set 119 and the console connector 116.

In various embodiments, the console connector 116 can further include a main body gasket 228. The main body gasket 228 can be configured to fit around a portion of the main body 220. In particular, the main body gasket 228 can be configured to fit around a neck portion 335 of the main body 220 near the main body proximal end 224. The main body gasket 228 can be configured to form a seal with an inner diameter of the cryoablation probe port 121 when the console connector 116 is inserted into the console 117.

In various embodiments, cryoablation probe assembly 103 can include one or more electrically conductive filaments or wires 336. In various embodiments, the wires 336 can be formed from a material or materials configured to transmit electrical signals (e.g., conductive wire). In such an embodiment, one or more sensors can be placed at the distal end of the shaft 104 of the cryoablation probe 101 and sensor data can be conveyed from the sensor(s) to the console via the electrically conductive filament. In one example, an electromagnetic position sensor can be included at the distal operating tip 108 of the shaft 104 and is configured to aid a physician in positioning the shaft within the patient's anatomy. In another example, compressive wrap wires could incorporate fiber optic cable(s) configured to provide information regarding the shape of the target anatomy. Any other suitable sensor or combination of sensors can be included at the distal end of the shaft including but not limited to temperature sensors, pressure sensors, strain sensors, or the like.

In various embodiments, the wires 336 are configured to connect the cryoablation probe 101 to the console 117. As best seen in FIG. 4, the wires 336 run from the cryoablation probe 101 to the proximal end of the line set 119 between the line set supply tube 112 and the line set return tube 114. An end of the wires 336 is configured to extend from the proximal end of the line set 119 and out through the main body 220 of the console connector 116. In various embodiments, the main body 220 is overmolded onto the wires 336 such that a proximal end of the wires protrudes from the main body of the console connector 116. In various embodiments, the overmolding of the main body 220 onto the wires 336 is configured to form a leak-tight seal around the wires and prevent the working fluid from leaking through the main body.

In various embodiments, the console connector 116 can further include a circuit board assembly 338. The circuit board assembly 338 can provide an electrical path from the wires 336 to an electrical adapter 339. The circuit board assembly 338 can include any suitable components such as a printed circuit board, or the like. In various embodiments, the proximal end of the wires 336 is configured to extend from the main body 220 of the console connector 116 and electrically connect to the circuit board assembly 338.

In various embodiments, the console connector 116 further includes an electrical cap 340 configured to cover the proximal end of the wires 336 and the circuit board assembly 338. The electrical cap 340 can be formed from any suitable material or materials, such as the same polymeric material as the main body 220. In various embodiments, the main body 220 includes an electrical adapter housing 350, which defines an opening for receiving the electrical adapter 339. In various embodiments, the electrical cap 340 can be joined to the electrical adapter housing 350 and the rest of the main body 220 by any suitable process such as a snap fit, a friction fit, adhesives, or the like.

In various embodiments, the console connector 116 further includes an electrical adapter 339 configured to electrically connect the one or more wires 336 to an electrical port of the console 117. In various embodiments, the electrical port of the console 117 can be located within or adjacent to the cryoablation probe port 121. Alternatively, the electrical port of the console 117 can be separate from the cryoablation probe port 121.

In various embodiments, the electrical adapter 339 can be accessed through an opening in the electrical cap 340. In the example of FIGS. 3-4, the electrical adapter 339 is a male 4-pin connector configured to mate with a female 4-pin connector in the console 117. Two pins of the 4-pin connector are visible in FIG. 4, which is a cross-section taken along a plan that bisects the electrical adapter 339. However, the electrical adapter 339 can be any suitable type of electrical adapter configured to electrically connect to a corresponding electrical port in the console 117.

In various embodiments, the console connector 116 can include a supply line adaptor 226 configured to engage with the main body 220 at the main body proximal end 224. In various embodiments, the supply line adaptor 226 can be formed from a polymeric material or materials, including, but not limited to polyimide, fluorinated ethylene propylene (FEP), Teflon, polytetrafluoroethylene (PTFE), or the like. In some embodiments, the supply line adaptor 226 can be formed from a resin, such as a Pebax based resin, an ABS based resin, a nylon-based resin, or the like. In some embodiments, the resin can contain glass-filled fibers that are configured to reduce shrinking and expansion of the supply line adaptor 226 caused by temperature fluctuations during a cryoablation procedure. In various embodiments, the supply line adaptor 226 is configured to sealingly connect to a proximal end of the supply tube 112 of the line set 119. In some embodiments, the supply line adaptor 226 is overmolded onto the proximal end of the supply tube 112. In various embodiments, overmolding the supply line adaptor 226 to the supply tube 112 provides a tight seal and prevents the working fluid from leaking from the interface between the line set 119 and the console connector 116.

In various embodiments, the console connector 116 can include a clip 334. In various embodiments the clip 334 is configured to fit around a portion of the supply line adaptor 226. In some embodiments, the clip 334 is configured to fit around a portion of the supply line adaptor 226 in an interference fit. In various embodiments, the clip 334 is configured to couple the supply line adaptor 226 to the main body 220. As can best be seen in FIG. 4, the clip 334 is configured to couple the supply line adaptor 226 to the main body 220 by fastening into a clip receiving structure 442 of the main body 220. In various embodiments, the clip receiving structure 442 of the main body 220 can include a cavity defined in the inner surface of the main body 220.

In various embodiments, the console connector 116 can further include a supply line adaptor gasket 341. The supply line adaptor gasket 341 can be configured to fit around a portion of the supply line adaptor 226. In particular, the supply line adaptor gasket 341 can be configured to fit around a neck portion or neck portion 337 (FIG. 3) of the supply line adaptor 226 near the supply line adaptor proximal end 227 (FIG. 2). The supply line adaptor gasket 341 can be configured to enhance the seal between the console connector 116 and the cryoablation probe port 121 when the console connector 116 is inserted into the console 117.

In various embodiments, to assemble the console connector 116, the supply line adaptor 226 is configured to be inserted into the main body 220 such that clip 334 engages with the clip receiving structure 442 of the main body 220. In some embodiments, the clip 334 engages with the clip receiving structure 442 of the main body 220 in an interference fit.

When the console connector 116 is in its assembled state, the supply line adaptor proximal end 227 extends proximally to the main body proximal end 224. The outer profile of the console connector 116 can contain a combination of protrusions, recesses, and gaskets which are configured to mate with and form a leak-tight seal with a corresponding set of mating features within the cryoablation probe port 121 of the console 117.

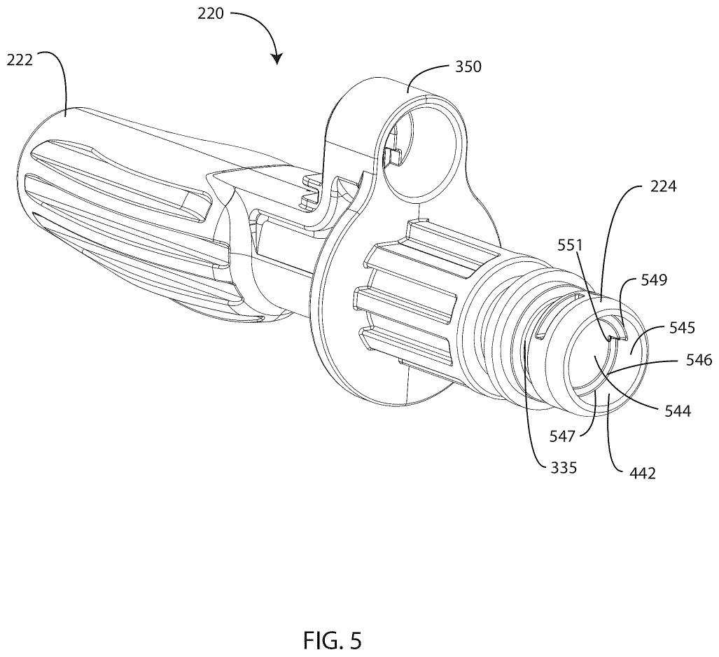

### Main Body (FIG. 5)

Referring now to FIG. 5, a perspective, schematic view of a main body of a probe-console connector is shown in accordance with various embodiments herein. In various embodiments, the main body 220 shown in FIG. 5 is an overmolded component, formed by being overmolded onto a proximal end of the return tube 114 (FIG. 4). In various embodiments, the main body 220 can include a main body distal end 222 that is configured to be connected via overmolding onto the proximal end of the return tube 114. The main body 220 can further define a neck portion 335 configured to receive the main body gasket 228. The main body 220 can further define an electrical adapter housing 350, which can receive the electrical adapter 339 (FIG. 4).

The main body can further include a main body proximal end 224. The main body proximal end can define an opening 544 configured to receive the supply line adaptor 226. The main body 220 can further define a clip receiving structure 442 configured to engage with the clip 334.

As can best be seen in FIG. 4, the clip 334 is configured to couple the supply line adaptor 226 to the main body 220 by fastening into a clip receiving structure 442 of the main body 220. In various embodiments, the clip receiving structure 442 can include a cavity surrounded by an inner surface 545 of the main body 220. In various embodiments, the clip receiving structure 442 can include a main body ledge 546 defined on the inner surface 545 of the main body 220. In various embodiments, the clip 334 is configured to engage with the inner surface 545 and main body ledge 546 of the main body 220.

In various embodiments, the main body ledge 546 can include a first ledge portion 547, a second ledge portion 549, and a ledge wall 551. In various embodiments, the first ledge portion 547 is disposed at a first depth from the proximal end of the main body 220 and the second ledge portion 549 is disposed at a second depth from the proximal end of the main body. In the example of FIG. 5, the first depth is greater than the second depth. In various embodiments, the clip 334 is configured to rest on the first ledge portion 547 and abut the second ledge portion 549 such that the ledge wall 551 is configured to prevent the clip from rotating with respect to the main body 220.

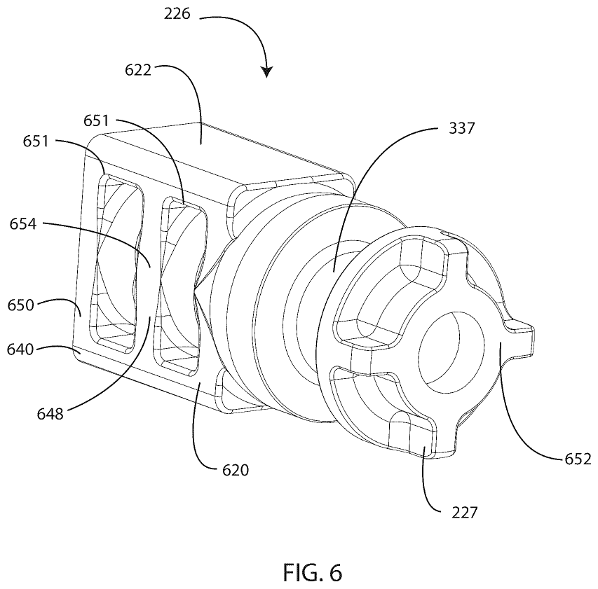

### Supply Line Adaptor (FIG. 6)

Referring now to FIG. 6, a perspective, schematic view of a supply line adaptor of a probe-console connector is shown in accordance with various embodiments herein. In various embodiments, the supply line adaptor 226 is attached to the proximal end of the supply tube by being overmolded onto the proximal end of the supply tube.

The supply line adaptor 226 can include a supply line adaptor proximal end 227. In various embodiments, supply line adaptor proximal end 227 can define a console plug piece 652. The console plug piece 652 can define a plurality of mating features configured to form a leak tight seal with a corresponding set of mating features within the cryoablation probe port 121 of the console 117. The supply line adaptor 226 can further define a neck portion 337 near the supply line adaptor proximal end 227 configured to receive the supply line adaptor gasket 341.

In various embodiments, the supply line adaptor 226 can include a distal portion 640 defining a clip receiving structure 648. In various embodiments the clip 334 is configured to fit around the clip receiving structure 648 of the supply line adaptor 226. In some embodiments, the clip 334 is configured to fit around the clip receiving structure 648 of the supply line adaptor 226 in an interference fit.

In various embodiments, the clip receiving structure 648 includes a first side 620, visible and facing the viewer in FIG. 6, and an opposite second side. The first side 620 and second side have identical structures in various embodiments. The first side 620 and second side of the clip receiving structure 648 can include a planar surface 650 and two openings 651 defined in the clip receiving structure 648. The two openings 651 are separated by a bridge portion 654 of the planar surface 650. In various embodiments, the two openings 651 are rectangular in shape.

In various embodiments, the clip receiving structure 648 further includes a third side 622 and an opposite fourth side. In various embodiments, the third side 622 and the fourth side include planar surfaces at right angles to the planar surfaces of the first and second sides.

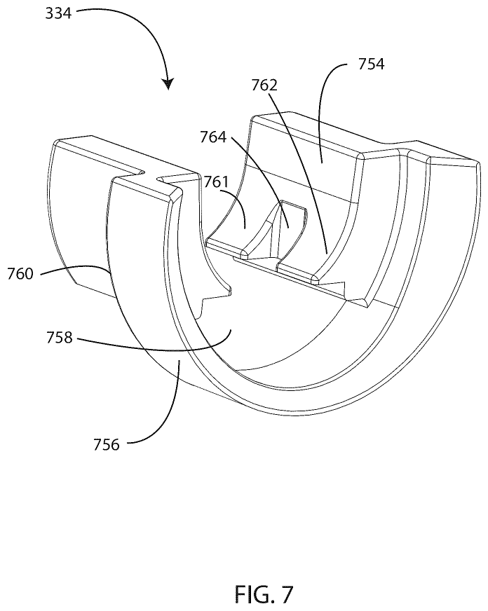

### Clip (FIG. 7)

Referring now to FIG. 7, a perspective, schematic view of a clip of a probe-console connector is shown in accordance with various embodiments herein. In various embodiments, the clip 334 can include a main body engagement structure 756. In various embodiments, the main body engagement structure 756 is configured to engage with the clip receiving structure 442 of the main body 220. In various embodiments, the clip 334 can include a clip ledge 760. The clip ledge 760 can be configured to engage with the main body ledge 546 when the clip is inserted into the main body 220.

In various embodiments, the clip 334 can include one or more relief features 758. The relief features 758 are configured to enhance the ductility of the clip 334. In some embodiments, the clip 334 can be compressed as it is inserted into the main body 220. Once inserted, the clip 334 can regain its original shape, creating an interference fit with the inner surface 545 and main body ledge 546 of the main body 220.

In various embodiments, the clip 334 can include supply line adaptor engagement structure 754. In various embodiments, the supply line adaptor engagement structure 754 is configured to engage with the clip receiving structure 648 of the supply line adaptor 226. In some embodiments, the clip 334 can be bent as it is inserted around the supply line adaptor 226. Once inserted, the clip 334 can regain its original shape, creating an interference fit with the clip receiving structure 648 of the supply line adaptor 226.

In various embodiments, the clip 334 includes a first half and a second half, where the second half is a mirror image of the first half. In various embodiments, each half of the clip 334 includes a supply line adaptor engagement structure 754. In various embodiments, the supply line adaptor engagement structure 754 includes a first ramp 761, a second ramp 762, and a notch 764 between the first ramp and second ramp. The first ramp and second ramp can snap into engagement with the two openings 651 of the clip receiving structure 648.

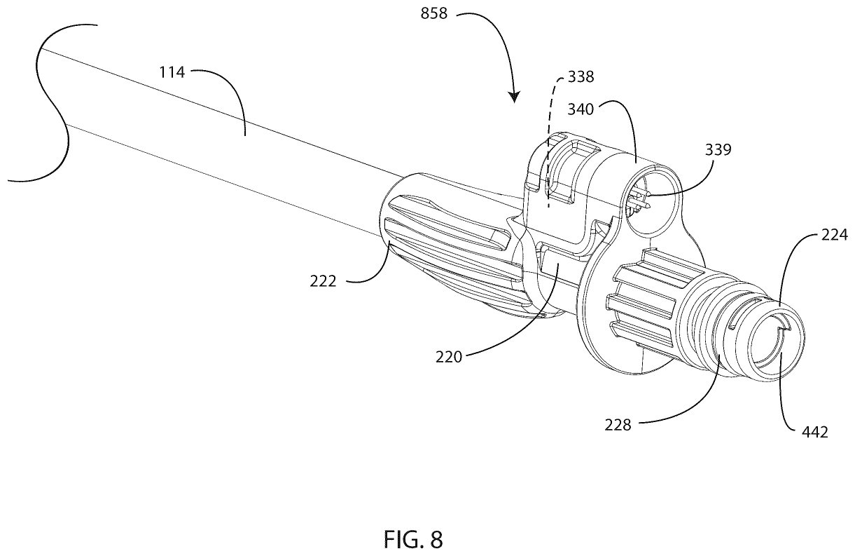

### Main Body Assembly (FIG. 8)

Referring now to FIG. 8, a perspective, schematic view of a main body assembly is shown in accordance with various embodiments herein. In various embodiments, to form the main body assembly 858, the main body 220 is overmolded onto the line set return tube 114. In some embodiments, the main body 220 is additionally overmolded onto one or more wires 336. The overmolding of the main body 220 is configured to form a leak-tight seal on the return flow of the working fluid and electrical wire pathways to ensure no leaks of the working fluid to the environment.

In various embodiments, the ends of the wires 336 exposed from the main body 220 are then electrically connected to the circuit board assembly 338. The circuit board assembly 338 is connected to the electrical adapter 339. The electrical cap 340 is then installed onto the main body 220 to cover the circuit board assembly and create a seamless profile. The main body gasket 228 can then be loaded onto the neck portion 335 of the main body to form a seal with an inner diameter of the cryoablation probe port 121 when the console connector 116 is inserted into the console 117.

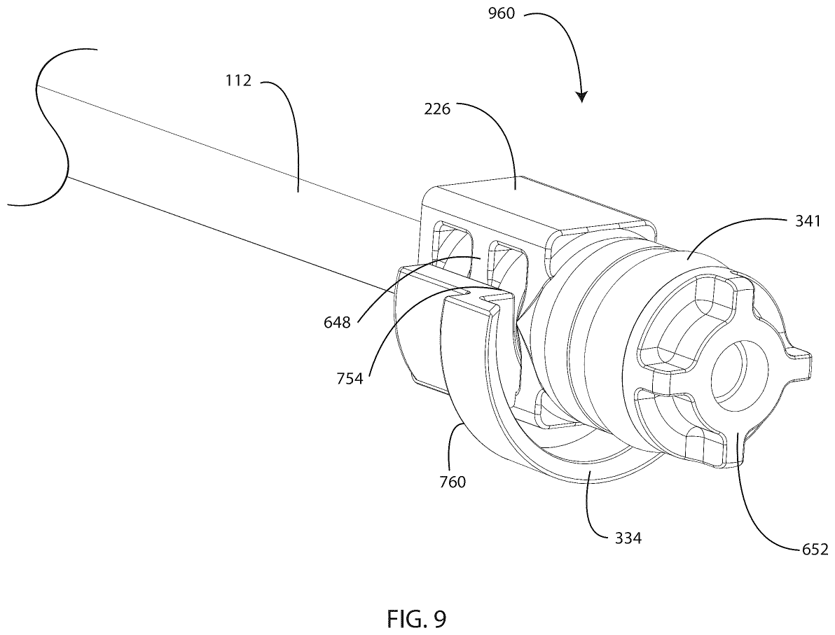

### Supply Line Adaptor Assembly (FIG. 9)

Referring now to FIG. 9, a perspective, schematic view of a supply line adaptor assembly is shown in accordance with various embodiments herein. In various embodiments, to form the supply line adaptor assembly 960, the supply line adaptor 226 is overmolded onto the line set supply tube 112. The supply line adaptor gasket 341 can then be loaded onto the neck portion 337 of the supply line adaptor 226 to enhance the seal between the console connector 116 and the cryoablation probe port 121 when the console connector 116 is inserted into the console 117.

In various embodiments, the clip 334 can then be attached to the supply line adaptor 226. In the example of FIG. 9, the clip 334 can snap around the supply line adaptor 226 such that the supply line adaptor engagement structure 754 of the clip is configured to engage with the clip receiving structure 648 of the supply line adaptor 226 in an interference fit.

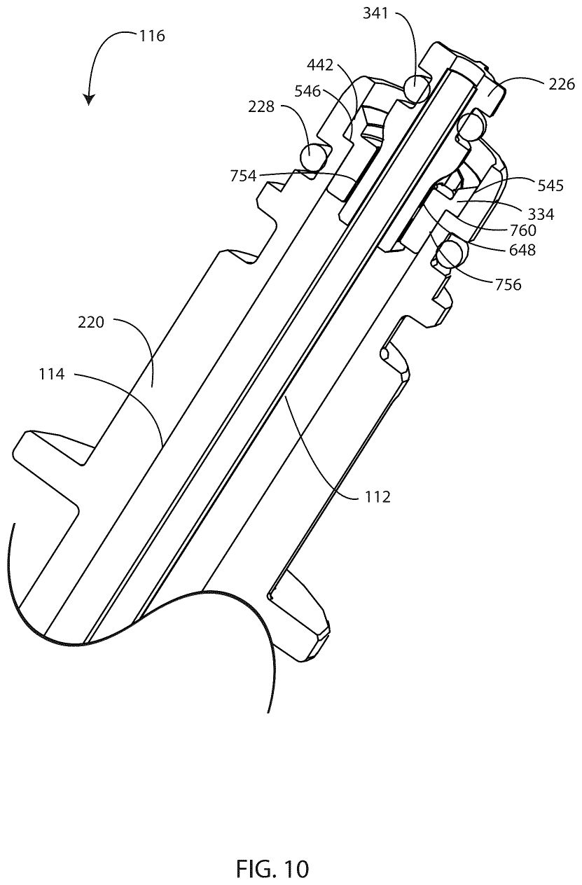

### Clip Within Main Body (FIG. 10)

Referring now to FIG. 10, a perspective view of a cross-section of the console connector is shown in accordance with various embodiments herein. In various embodiments, the clip 334 is configured to snap around the supply line adaptor 226 such that the supply line adaptor engagement structure 754 of the clip is configured to engage with the clip receiving structure 648 of the supply line adaptor 226. In some embodiments, the clip 334 can be bent as it is inserted around the supply line adaptor 226. Once inserted, the clip 334 can regain its original shape, creating an interference fit with the clip receiving structure 648 of the supply line adaptor 226.

Upon attaching the clip 334 to the supply line adaptor 226, the console connector can be assembled by inserting the supply line adaptor assembly into the main body 220 such that clip 334 engages with the clip receiving structure 442 of the main body 220. The main body proximal end can define an opening 544 configured to receive the supply line adaptor 226. The main body 220 can further define clip receiving structure 442 configured to engage with the clip 334.

In various embodiments, the main body engagement structure 756 of the clip 334 is configured to engage with the clip receiving structure 442 of the main body 220. In various embodiments, the clip 334 can include a clip ledge 760. The clip ledge 760 can be configured to engage with the main body ledge 546 when the clip is inserted into the main body 220. In various embodiments, the engagement between the clip ledge 760 and main body ledge 546 is configured to keep the clip suspended in place within the main body 220.

In some embodiments, the main body engagement structure 756 of the clip 334 is configured to form an interference fit with the clip receiving structure 442 of the main body 220, preventing motion of the clip 334 relative to the main body 220. In some embodiments, the main body engagement structure 756 of the clip 334 is configured to key into the clip receiving structure 442 of the main body 220, preventing motion of the clip 334 relative to the main body 220.

The cross section depicted by FIG. 10 is taken at the boundary between the first ledge portion 547 and the second ledge portion 549 of the main body ledge 546. In some embodiments, the clip ledge 760 is configured to rest on the first ledge portion 547 of the main body ledge 546 and abut the second ledge portion 549 of the main body ledge such that the ledge wall 551 is configured to prevent the clip 334 from rotating with respect to the main body 220.

In some embodiments, the clip 334 can be compressed as it is inserted into the main body 220. Once inserted, the clip 334 can regain its original shape, creating an interference fit with the inner surface 545 and main body ledge 546 of the main body 220.

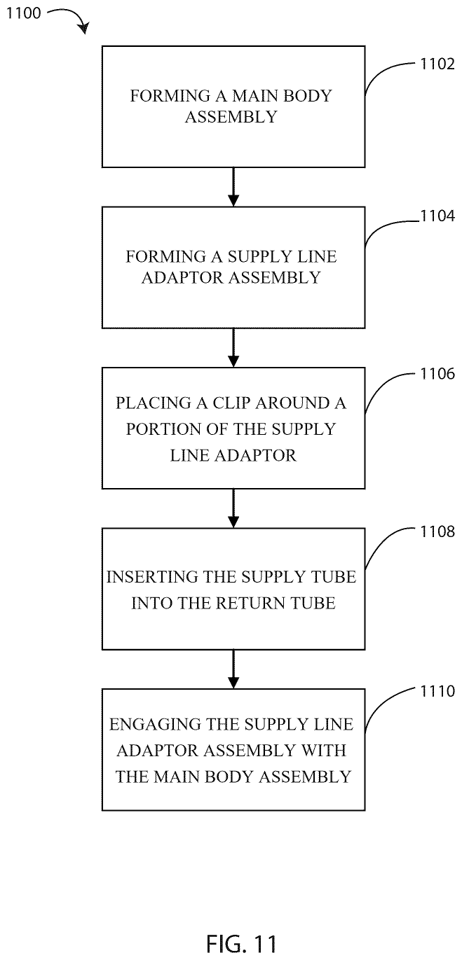

### Method of Forming a Console Connector (FIG. 11)

Many different methods are contemplated herein, including, but not limited to, methods of making, methods of using, and the like. Aspects of system/device operation described elsewhere herein can be performed as operations of one or more methods in accordance with various embodiments herein. Referring now to FIG. 11, a method 1100 of forming a console connector configured to sealingly connect a cryoablation probe assembly to a cryoablation probe port of a console is shown in accordance with various embodiments herein.

In various embodiments, the method 1100 can include a step 1102 of forming a main body assembly. As shown and described by FIG. 8, to form the main body assembly 858, a main body 220 of the console connector 116 is overmolded onto the proximal end of a line set return tube 114. In some embodiments, step 1102 can include overmolding the main body 220 onto one or more wires 336.

In some embodiments, step 1102 can include electrically connecting the wires 336 to a circuit board assembly 338 and connecting the circuit board assembly 338 to an electrical adapter 339. The electrical adapter 339 can be positioned within an electrical adapter housing 350. In some embodiments, step 1102 can include covering the wires 336 and circuit board assembly with an electrical cap 340. In some embodiments, step 1102 can include attaching the electrical cap 340 onto the main body. In some embodiments, step 1102 can include loading a main body gasket 228 onto a neck portion 335 of the main body 220.

In various embodiments, the method 1100 can include a step 1104 of forming a supply line adaptor assembly. As shown and described by FIG. 9, to form the supply line adaptor assembly 960, the supply line adaptor 226 is overmolded onto the proximal end of a line set supply tube 112. In some embodiments, step 1104 can include loading the supply line adaptor gasket 341 onto the neck portion 337 of the supply line adaptor 226.

In various embodiments, the method 1100 can include a step 1106 of placing a clip around a portion of the supply line adaptor. In some embodiments, step 1106 can include snapping the clip 334 around the supply line adaptor 226 such that the supply line adaptor engagement structure 754 of the clip is configured to engage with the clip receiving structure 648 of the supply line adaptor 226 in an interference fit.

In various embodiments, the method 1100 can include a step 1108 of inserting the supply tube 112 into the return tube 114. In various embodiments, step 1108 can include inserting a distal end of the supply tube 112 into a proximal end of the return tube 114. In alternative embodiments, step 1108 can include inserting a proximal end of the supply tube 112 into a distal end of the return tube 114. In some embodiments, step 1108 can include inserting the supply line adaptor assembly 960 into the main body assembly 858 via the opening 544 defined in the main body proximal end.

In various embodiments, the method 1100 can include a step 1110 of engaging the supply line adaptor assembly with an inner diameter of the main body such that the clip fastens into a clip receiving structure of an inner surface of the main body. In some embodiments, step 1110 can include engaging the main body engagement structure 756 of the clip 334 with the clip receiving structure 442 of the main body 220. In some embodiments, step 1110 can include engaging the clip ledge 760 with the main body ledge 546. In some embodiments, step 1110 can include compressing the clip 334 as it is inserted into the main body 220. In some embodiments, step 1110 can include allowing the clip 334 to regain its original shape, creating an interference fit with the inner surface 545 and main body ledge 546 of the main body 220.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cryosurgery system, comprising:
a cryoablation probe assembly, the cryoablation probe assembly comprising a supply tube, and a return tube surrounding the supply tube;
a console comprising a cryoablation probe port;
a console connector configured to sealingly connect the cryoablation probe assembly to the cryoablation probe port of the console, the console connector comprising:
a main body configured to sealingly connect to a proximal end of the return tube; and
a supply line adaptor configured to sealingly connect to a proximal end of the supply tube; and
a clip, wherein the clip is configured to couple the supply line adaptor to the main body by fastening into a clip receiving structure of an inner surface of the main body.

2. The cryosurgery system of claim 1, wherein the main body is overmolded onto the proximal end of the return tube.

3. The cryosurgery system of claim 1, wherein the supply line adaptor is overmolded onto the proximal end of the supply tube.

4. The cryosurgery system of claim 1, the console connector further comprising a circuit board assembly, wherein the main body is configured to be overmolded onto one or more wires connected to the cryoablation probe assembly, wherein a proximal end of each of the one or more wires protrudes from the main body and connects to the circuit board assembly.

5. The cryosurgery system of claim 4, wherein the console connector further comprises an electrical cap configured to cover the circuit board assembly.

6. The cryosurgery system of claim 5, wherein the circuit board assembly comprises an adaptor configured to electrically connect the one or more wires of the console connector to the cryoablation probe port of the console.

7. The cryosurgery system of claim 1, wherein the cryosurgery system is a closed-loop cryosurgery system.

8. The cryosurgery system of claim 1, wherein the console connector further comprises a main body gasket, wherein the main body gasket is configured to form a seal with an inner diameter of the cryoablation probe port.

9. The cryosurgery system of claim 1, wherein the console connector further comprises a supply line adaptor gasket configured to be positioned at a neck of a supply line adaptor proximal end.

10. The cryosurgery system of claim 1, wherein the clip is configured to fit around a portion of the supply line adaptor in an interference fit.

11. The cryosurgery system of claim 10, wherein the clip receiving structure of the main body comprises a cavity defined in the inner surface of the main body.

12. The cryosurgery system of claim 1, wherein the main body and the supply line adaptor comprise a polymeric material.

13. A console connector configured to sealingly connect a cryoablation probe assembly to a cryoablation probe port of a console, the console connector comprising:
a main body configured to sealingly connect to a proximal end of a return tube of the cryoablation probe assembly; and
a supply line adaptor assembly comprising:
a supply line adaptor configured to sealingly connect to a proximal end of a supply tube of the cryoablation probe assembly; and
a clip, wherein the clip is configured to couple the supply line adaptor to the main body by fastening into a clip receiving structure of an inner surface of the main body; and
wherein the supply tube is positioned within the return tube when the supply line adaptor is fit within the main body.

14. The console connector of claim 13, wherein the main body is overmolded onto the proximal end of the return tube and wherein the supply line adaptor is overmolded onto the proximal end of the supply tube.

15. The console connector of claim 13, wherein the main body is configured to be overmolded onto one or more wires connected to the cryoablation probe, and wherein a proximal end of each of the one or more wires protrudes from the main body and connects to a circuit board assembly.

16. The console connector of claim 13, the console connector further comprises a main body gasket, wherein the main body gasket is configured to form a seal with the cryoablation probe port.

17. The console connector of claim 13, the supply line adaptor assembly further comprising a supply line adaptor gasket configured to engage with a neck feature of a supply line adaptor proximal end.

18. The console connector of claim 13, the clip is configured to fit around a portion of the supply line adaptor in an interference fit.

19. The console connector of claim 13, wherein the main body and the supply line adaptor comprise a polymeric material.

20. A method of forming a console connector configured to sealingly connect a cryoablation probe assembly to a cryoablation probe port of a console comprising:
providing a return tube;
forming a main body assembly by overmolding a main body onto a proximal end of the return tube, wherein the main body is sealingly connected to the proximal end of a return tube;
providing a supply tube configured to fit within the return tube;
forming a supply line adaptor assembly by:
overmolding a supply line adaptor onto a proximal end of the supply tube, wherein the supply line adaptor is sealingly connected to the proximal end of the supply tube; and
placing a clip around a portion of the supply line adaptor;
inserting the supply tube into the return tube; and
engaging the supply line adaptor assembly with an inner diameter of the main body such that the clip fastens into a clip receiving structure of an inner surface of the main body.
